(19) United States
(12) Patent Application Publication
(10) Pub. No.: US 2026/0315015 A1
(43) Pub. Date: Oct. 15, 2026

(54) Systems and Methods for Confirming Position or Orientation of Medical Device Relative to Target

(71) Applicant: Covidien LP, Mansfield, MA (US)

(72) Inventors: Guy ALEXANDRONI, Yehud-Monosson (IL); Ruth LIBKIND, Tel Aviv (IL)

(21) Appl. No.: 19/116,535

(22) PCT Filed: Oct. 10, 2023
(86) PCT No.: PCT/IB2023/060188

§ 371 Date: Mar. 28, 2025

Related U.S. Application Data
(60) Provisional application No. 63/416,329, filed on Oct. 14, 2022.

(51) Int. Cl. A61B 6/12; A61B 6/00; A61B 6/46; A61B 10/04; G06T 7/73; G06T 19/00

(52) U.S. Cl. CPC A61B 6/12; A61B 6/463; A61B 6/468; A61B 6/5235; A61B 10/04; G06T 7/74; G06T 19/00; G06T 2207/10016; G06T 2207/10081; G06T 2207/30061; G06T 2210/41

(57) ABSTRACT

Systems and methods for visually verifying whether a medical device is inside or pointing towards a target use intraoperative imaging while the medical device is at or near the target. The systems and methods involve performing a fluoroscopic sweep of a patient in which a medical device is placed, reconstructing a volume based on the fluoroscopic sweep, displaying an initial slice of the volume from which a user starts a search, and receiving information identifying the medical device's tip and the target in the volume as the user scrolls through slices of the volume. Scrolling through the volume allows the user to ascertain the relationship between the medical device and the target. Alternatively, feedback is provided to the user by augmenting the markings of the medical device's tip and the target and/or notifying the user whether the medical device's tip is inside or pointing towards the target.

### FIELD

The technology is generally related to systems and methods of navigation and position or orientation confirmation for surgical procedures. More particularly, this disclosure relates to systems and methods for confirming a position or orientation of a medical device relative to a target using two dimensional intraoperative X-ray images captured using a standard X-ray imaging device and a three dimensional volume constructed from the two dimensional intraoperative X-ray images.

### BACKGROUND

There are several commonly applied methods for treating various maladies affecting organs including the liver, brain, heart, lung, and kidney. Often, one or more imaging modalities, such as magnetic resonance imaging, ultrasound imaging, computed tomography (CT), as well as others are employed by clinicians to identify areas of interest within a patient and ultimately targets for treatment.

An endoscopic approach has proven useful in navigating to areas of interest within a patient, and particularly so for areas within luminal networks of the body such as the lungs. To enable the endoscopic, and more particularly the bronchoscopic, approach in the lungs, endobronchial navigation systems have been developed that use previously acquired MRI data or CT image data to generate a three dimensional rendering or volume of the particular body part such as the lungs. In particular, previously acquired images, acquired from an MRI scan or CT scan of the patient, are utilized to generate a three dimensional or volumetric rendering of the particular body part of the patient.

The resulting volume generated from the MRI scan or CT scan is then utilized to create a navigation plan to facilitate the advancement of a navigation catheter (or other suitable device) through a bronchoscope and a branch of the bronchus of a patient to an area of interest. Electromagnetic tracking may be utilized in conjunction with the CT data to facilitate guidance of the navigation catheter through the branch of the bronchus to the area of interest. In certain instances, the navigation catheter may be positioned within one of the airways of the branched luminal networks adjacent to, or within, the area of interest to provide access for one or more medical instruments.

Thus, in order to generate a navigation plan, or in order to even generate a three dimensional or volumetric rendering of the patient's anatomy, such as the lung, a clinician is required to utilize an MRI system or CT system to acquire the necessary image data for construction of the three dimensional volume. An MRI system or CT-based imaging system is extremely costly and, in many cases, is not available in the same location as the location where a navigation plan is generated or where a navigation procedure is carried out.

A fluoroscopic imaging device is commonly located in the operating room during navigation procedures. A clinician may use the standard fluoroscopic imaging device to visualize and confirm the placement of a tool after it has been navigated to a desired location. However, although standard fluoroscopic images display highly dense objects such as metal tools and bones as well as large soft-tissue objects such as the heart, the fluoroscopic images have difficulty resolving small soft-tissue objects of interest such as lesions. Further, the fluoroscope image is only a two dimensional projection. In order to be able to see small soft-tissue objects in three dimensional space, an X-ray volumetric reconstruction is needed. Several solutions exist that provide three dimensional volume reconstruction of soft-tissues such as CT and Cone-beam CT which are extensively used in the medical world. These machines algorithmically combine multiple X-ray projections from known, calibrated X-ray source positions into three dimensional volume in which the soft tissues are visible.

In order to navigate tools to a remote soft-tissue target for biopsy or treatment, both the tool and the target should be visible in some sort of a three dimensional guidance system. The majority of these systems use some X-ray device to see through the body. For example, a CT machine can be used with iterative scans during procedure to provide guidance through the body until the tools reach the target. This is a tedious procedure as it requires several full CT scans, a dedicated CT room and blind navigation between scans. In addition, each scan requires the staff to leave the room. Another option is a Cone-beam CT machine which is available in some operation rooms and is somewhat easier to operate, but is expensive and like the CT only provides blind navigation between scans, requires multiple iterations for navigation, and requires the staff to leave the room.

### SUMMARY

The techniques of this disclosure generally relate to systems and methods for confirming whether a medical device is inside and/or aligned with a target using intraoperative imaging while the medical device is at or near a target.

In one aspect, the disclosure provides a system including a computer system having a processor and a display that displays a graphical user interface. The computer system also has a computer readable storage medium storing thereon instructions that when executed by the processor cause the processor to receive a sequence of intraoperative X-ray images. Each intraoperative X-ray image includes at least a portion of a medical device and a target. The instructions, when executed by the processor, also cause the processor to receive a marking of a tip of the medical device in at least two of the sequence of intraoperative X-ray images and construct a three-dimensional (3D) reconstruction based on the sequence of intraoperative X-ray images and the markings of the tip of the medical device, the 3D reconstruction including the medical device and the target. The instructions, when executed by the processor, also cause the processor to display a slice of the 3D reconstruction passing through the tip of the medical device, determine a position of the target in the 3D reconstruction; and present feedback on the position of the tip of the medical device relative to the target.

Implementations of the system may also include one or more of the following features. The instructions, when executed by the processor, may cause the processor to augment the marking of the target or the marking of the medical device to show that the medical device is inside or outside the target. The instructions, when executed by the processor, may cause the processor to present a message indicating that the medical device is inside or outside the target. The instructions, when executed by the processor, may cause the processor to determine that the tip of the medical device is outside of the target, in response to determining that the tip of the medical device is outside of the target, determine whether the tip of the medical device is aligned with the target, and present a message indicating whether the tip of the medical device is aligned with the target. The instructions, when executed by the processor, may cause the processor to determine that the tip of the medical device is outside of the target, in response to determining that the tip of the medical device is outside of the target, determine a distance of the tip of the medical device from the target or the position of the tip of the medical device relative to the target, and present a message indicating the distance of the tip of the medical device from the target or the position of the tip of the medical device relative to the target.

In aspects, the instructions, when executed by the processor, may cause the processor to receive preoperative computed tomography (CT) images of the target, construct a 3D model of the target based on the preoperative CT images, and overlay the 3D model of the target on the 3D reconstruction. The instructions, when executed by the processor, may cause the processor to register the preoperative CT images with the 3D reconstruction, determine a position of the target in the 3D reconstruction based on the registering yielding a determined position of the target, and overlay the 3D model of the target on the 3D reconstruction based on the determined position of the target.

In aspects, the instructions, when executed by the processor, may cause the processor to construct a 3D model of the medical device based on the sequence of intraoperative X-ray images, and overlay the 3D model of the medical device on the 3D reconstruction. The instructions, when executed by the processor, may cause the processor to receive preoperative computed tomography (CT) images of the target; segment the target from the preoperative CT images, yielding a segmented target, and overlay the segmented target on the 3D reconstruction. The instructions, when executed by the processor, may cause the processor to receive preoperative computed tomography (CT) images of a lung, receive at least one marking of the target on the preoperative CT images, and overlay the at least one marking of the target on the 3D reconstruction. The at least one marking of the target may represent a size or a shape of the target.

In aspects, the sequence of intraoperative X-ray images may be fluoroscopic images or cone beam CT images. The medical device may not include a position sensor. The medical device may be a biopsy tool.

In another aspect, the disclosure provides a method. The method includes receiving a sequence of intraoperative X-ray images, receiving a marking of a tip of a medical device in at least two of the sequence of intraoperative X-ray images, and generating a three-dimensional (3D) reconstruction based on the sequence of intraoperative X-ray images and the markings of the tip of the medical device. The 3D reconstruction includes the at least a portion of the medical device and a target. The method also includes displaying a slice of the 3D reconstruction passing through the tip of the medical device, determining a position of the target in the 3D reconstruction, and presenting feedback on the position of the tip of the medical device relative to the target.

Implementations of the method may also include one or more of the following features. The method may include augmenting the marking of the target or the marking of the medical device to show that the medical device is inside or outside the target. The method may include presenting a message indicating that the medical device is inside or outside the target.

The method may include determining that the tip of the medical device is outside of the target, determining that the tip of the medical device is aligned with the target in response to determining that the tip of the medical device is outside of the target, determining a distance of the tip of the medical device from the target in response to determining that the tip of the medical device is aligned with the target, and presenting a message indicating that the tip of the medical device is aligned with the target and the distance of the tip of the medical device from the target.

The method may include determining that the tip of the medical device is outside of the target, determining that the tip of the medical device is not aligned with the target in response to determining that the tip of the medical device is outside of the target, determining the position of the tip of the medical device relative to the target in response to determining that the tip of the medical device is not aligned with the target, and presenting a message indicating that the tip of the medical device is not aligned with the target and the position of the tip of the medical device relative to the target.

In still another aspect, the disclosure provides another system including a computer system having a processor and a display that display a graphical user interface. The computer system also has a computer readable storage medium storing thereon instructions that when executed by the processor cause the processor to receive a sequence of intraoperative X-ray images from an X-ray imaging device. Each intraoperative X-ray image includes at least a portion of a medical device and a target. The instructions, when executed by the processor, also cause the processor to estimate a pose of the X-ray imaging device based on the sequence of intraoperative X-ray images yielding an estimated pose and generate a three-dimensional (3D) volume based on the sequence of intraoperative X-ray images and the estimated pose. The 3D volume includes the at least a portion of the medical device and the target. The instructions, when executed by the processor, also cause the processor to display a slice of the 3D volume, from which a user starts a search for a tip of the medical device and the target in the slices of the 3D volume, receive position information of a scroll control object, and display other slices of the 3D volume corresponding to the position information.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

Various aspects and embodiments of the disclosure are described hereinbelow with references to the drawings, wherein:

FIG. 1 is a perspective view of one illustrative example of an electromagnetic navigation (EMN) system incorporating a fluoroscopic imaging device in accordance with the disclosure;

FIG. 2A is a diagram that illustrates a user interface showing a slice of a fluoroscopic 3D reconstruction centered at the target;

FIG. 2B is a diagram that illustrates a user interface showing a slice of the fluoroscopic 3D reconstruction centered at the tip of the medical device;

FIGS. 3 and 4 are flowcharts that illustrate methods of visualizing the relationship between a medical device's tip and a target;

FIG. 5 is a diagram that illustrates a user interface for marking a medical device's tip in fluoroscopic images of a portion of a lung;

FIG. 6 is a diagram that illustrates a user interface for displaying a reconstructed volume and marking the target;

FIG. 7 is a diagram that illustrates a user interface for displaying an example of an augmentation to the marking of the medical device's tip;

FIG. 8 is a flowchart that illustrates another method of visualizing the relationship between a medical device's tip and a target;

FIG. 9 is a diagram that illustrates a user interface for providing textual feedback to a user while displaying a reconstructed volume in accordance with the method of FIG. 8;

FIGS. 10A-10H are diagrams that illustrate other examples of user interfaces that implement aspects of the methods described herein; and

FIG. 11 is a diagram that illustrates a system configured for use with the methods of the disclosure.

### DETAILED DESCRIPTION

In navigating a medical device to a target, a fluoroscopic three dimensional reconstruction or volume may be generated from two dimensional fluoroscopic images using limited angle tomosynthesis and displayed to help the clinician align the medical device with the target or confirm that the tip of the medical device is within the target. However, there is significant scattering in the Anterior-Posterior (AP) direction. The scattering makes it challenging to determine visually if the tip of the medical device (e.g., a biopsy tool) is inside the target (e.g., a lesion), above the target, or below the target. When using a CT-like volume this challenge does not exist. Also, the 3D visualization of the target may significantly deteriorate in a case of a biopsy procedure that leads to local bleeding and atelectasis.

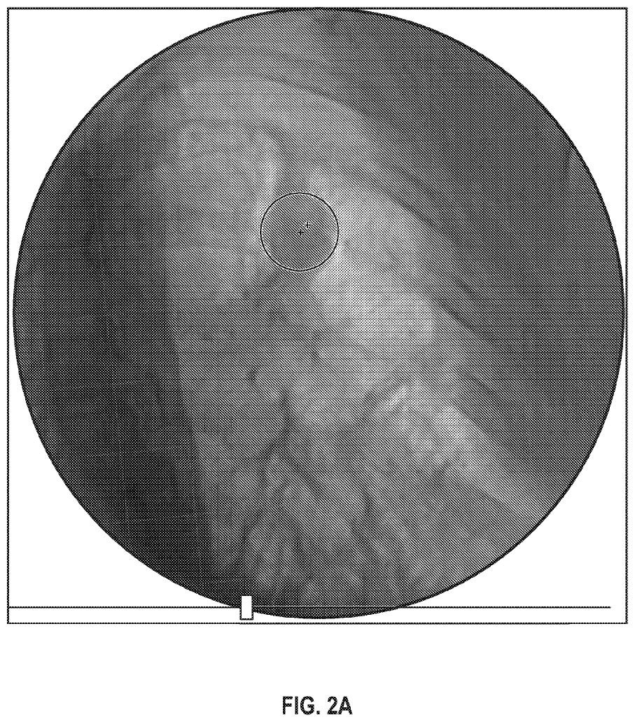

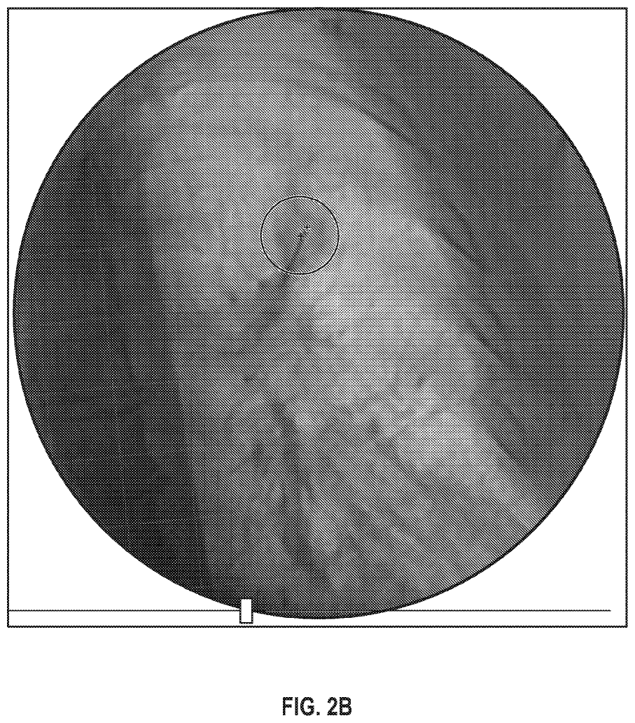

FIGS. 2A and 2B illustrate the challenge of understanding whether a medical device is above or below a target by scrolling through slices of a fluoroscopic 3D reconstruction. FIG. 2A shows a slice of the fluoroscopic 3D reconstruction centered at the target and FIG. 2B shows a slice of the fluoroscopic 3D reconstruction centered at the tip of the medical device. As shown in FIG. 2B, the target is still visible at the slice centered at the tool's tip. However, based on the target radius from the planning, the tool is slightly below the target.

This disclosure is directed to a system and method that supports the clinician in deciding whether a medical procedure (e.g., a biopsy) is performed at the correct location, by providing visual confirmation to the clinician that an end portion of a medical device (e.g., a biopsy tool) is inside or pointing towards the target. The confirmation may be provided via intra-operative imaging while the end portion of the medical device is in the vicinity of the target.

The systems and methods of the disclosure are performed after navigating a medical device near a target and employ a 3D reconstruction generated based on intraoperative fluoroscopic images using limited angle tomosynthesis. The systems and methods of the disclosure also eliminate dependency on a locatable guide (LG) so that the medical device (e.g., biopsy tool) can be placed at a location for performing a medical procedure (e.g., a biopsy).

The systems and methods include performing a sweep with a fluoroscope, which yields a sequence of fluoroscopic images, reconstructing a volume based on the sequence of fluoroscopic images, displaying an initial slice from which the clinician starts a search, and identifying the target and medical device's tip in the reconstructed volume by enabling the clinician to scroll through slices of the reconstructed volume. In some aspects, the relationship between the medical device and the target may be displayed such that the clinician can understand the relationship between the medical device and the target, e.g., that the tip of the medical device is inside or aligned with the target.

Alternatively, in other aspects, additional visualization features are provided as feedback to the clinician. For example, the markings of the target and/or medical device's tip are augmented and/or a message or notification is provided to the clinician indicating whether the tip of the medical device is inside or outside of the target. In aspects, the systems and methods of the disclosure estimate the orientation of the medical device's tip, forecast where the medical device's tip would travel if the clinician advanced the medical device along a trajectory from the current position of the medical device's tip and aligned with the orientation of the medical device's tip, then display the trajectory, which may also help the clinician understand if the end portion of the medical device is aligned with the target.

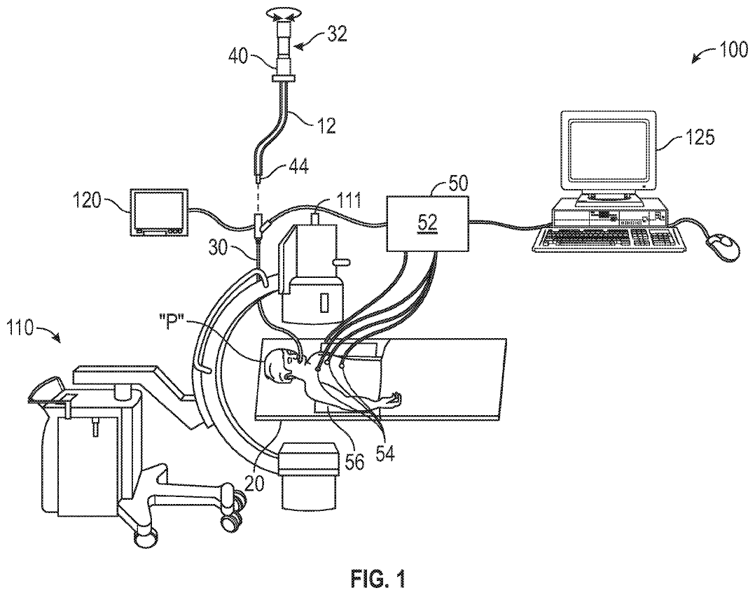

FIG. 1 depicts an Electromagnetic Navigation (EMN) system 100 configured for reviewing CT image data to identify one or more targets, planning a pathway to an identified target (planning phase), navigating an extended working channel (EWC) 12 of a catheter assembly to a target (navigation phase) via a user interface, and confirming placement of the EWC 12 and a medical instrument (e.g., a biopsy tool) relative to the target. One such EMN system is the ELECTROMAGNETIC NAVIGATION BRONCHOSCOPY® system currently sold by Medtronic PLC. The target may be tissue of interest identified by review of the CT image data during the planning phase. Following navigation, a medical device, such as a biopsy tool or other tool, may be inserted into the EWC 12 to obtain a tissue sample from tissue located at, or proximate to, the target.

As shown in FIG. 1, the EWC 12 is part of a catheter guide assembly 40. In practice, the EWC 12 is inserted into bronchoscope 30 for access to a luminal network of the patient P. Specifically, the EWC 12 of catheter guide assembly 40 may be inserted into a working channel of bronchoscope 30 for navigation through a patient's luminal network. A locatable guide (LG) 32, including a sensor 44 is inserted into the EWC 12 and locked into position such that the sensor 44 extends a desired distance beyond the distal tip of the EWC 12. The position and orientation of the sensor 44 relative to the reference coordinate system, and thus the distal portion of the EWC 12, within an electromagnetic field can be derived. Catheter guide assemblies 40 are currently marketed and sold by Medtronic PLC under the brand names superDimension™ Procedure Kits, or EDGE™ Procedure Kits, and are contemplated as useable with the disclosure. For a more detailed description of the catheter guide assemblies 40, reference is made to commonly-owned U.S. Patent Publication No. 2014/0046315, filed on Mar. 15, 2013, by Ladtkow et al, U.S. Pat. Nos. 7,233,820, and 9,044,254, the entire contents of each of which are hereby incorporated by reference.

The EMN system 100 generally includes an operating table 20 configured to support a patient P, a bronchoscope 30 configured for insertion through the patient's P's mouth into the patient's P's airways; monitoring equipment 120 coupled to the bronchoscope 30 (e.g., a video display, for displaying the video images received from the video imaging system of the bronchoscope 30); a tracking system 50 including a tracking module 52, a plurality of reference sensors 54 and a transmitter mat 56; and a workstation or computer system 125 including software and/or hardware used to facilitate identification of a target, pathway planning to the target, navigation of a medical device to the target, confirmation of placement of an EWC 12, and confirmation of placement of a medical device, which extends through and out of the EWC 12, relative to the target.

A fluoroscopic imaging device 110 capable of acquiring fluoroscopic or x-ray images or video of the patient P is also included in this particular aspect of the system 100. The images, series of images, or video captured by the fluoroscopic imaging device 110 may be stored within the fluoroscopic imaging device 110 or transmitted to the computer system 125 for storage, processing, and display. Additionally, the fluoroscopic imaging device 110 may move relative to the patient P so that images may be acquired from different angles or perspectives relative to the patient P to create a fluoroscopic video. In one aspect, the fluoroscopic imaging device 110 includes an angle measurement device 111 which is configured to measure the angle of the fluoroscopic imaging device 110 relative to the patient P. The angle measurement device 111 may be an accelerometer. The fluoroscopic imaging device 110 may include a single imaging device or more than one imaging device. In the case where the fluoroscopic imaging device 110 includes multiple imaging devices, each imaging device may be a different type of imaging device or the same type. Further details regarding the fluoroscopic imaging device 110 are described in U.S. Pat. No. 8,565,858, which is incorporated by reference in its entirety herein.

The computer system 125 may be any suitable computer system including a processor and storage medium, wherein the processor is capable of executing instructions stored on the storage medium. The computer system 125 may further include a database configured to store patient data, CT data sets including CT images, fluoroscopic data sets including fluoroscopic images and video, navigation plans, and any other such data. Although not explicitly illustrated, the computer system 125 may include inputs, or may otherwise be configured to receive, CT data sets, fluoroscopic images or video, and other data described herein. Additionally, the computer system 125 includes a display configured to display graphical user interfaces. The computer system 125 may be connected to one or more networks through which one or more databases may be accessed.

With respect to the planning phase, the computer system 125 utilizes previously acquired CT image data for generating and viewing a three dimensional model of the patient's P's airways, enables the identification of a target on the three dimensional model (automatically, semi-automatically, or manually), and allows for determining a pathway through the patient's P's airways to tissue located at and around the target. More specifically, CT images acquired from previous CT scans are processed and assembled into a three dimensional CT volume, which is then utilized to generate a three dimensional model of the patient's P's airways. The three dimensional model may be displayed on a display associated with the computer system 125, or in any other suitable fashion. Using the computer system 125, various views of the three dimensional model or enhanced two dimensional images generated from the three dimensional model are presented. The enhanced two dimensional images may possess some three dimensional capabilities because they are generated from three dimensional data. The three dimensional model may be manipulated to facilitate identification of target on the three dimensional model or two dimensional images, and selection of a suitable pathway through the patient's P's airways to access tissue located at the target can be made. Once selected, the pathway plan, the three dimensional model, and the images derived therefrom, can be saved and exported to a navigation system for use during the navigation phase(s). One such planning software is the superDimension™M planning suite currently sold by Medtronic PLC.

With respect to the navigation phase, a six degrees-of-freedom electromagnetic tracking system 50, e.g., similar to those disclosed in U.S. Pat. Nos. 8,467,589, 6,188,355, and published PCT Application Nos. WO 00/10456 and WO 01/67035, the entire contents of each of which are incorporated herein by reference, or other suitable positioning measuring system, is utilized for performing registration of the images and the pathway for navigation, although other configurations are also contemplated. The tracking system 50 includes a tracking module 52, a plurality of reference sensors 54, and a transmitter mat 56. The tracking system 50 is configured for use with a locatable guide 32 and particularly the sensor 44. As described above, the locatable guide 32 and the sensor 44 are configured for insertion through an EWC 12 into a patient's P's airways (either with or without the bronchoscope 30) and are selectively lockable relative to one another via a locking mechanism.

The transmitter mat 56 is positioned beneath patient P. The transmitter mat 56 generates an electromagnetic field around at least a portion of the patient P within which the position of the reference sensors 54 and the sensor 44 can be determined with use of a tracking module 52. One or more of the reference sensors 54 are attached to the chest of the patient P. The six degrees of freedom coordinates of the reference sensors 54 are sent to the computer system 125 (which includes the appropriate software) where they are used to calculate a patient coordinate frame of reference. Registration, as detailed below, is generally performed to coordinate locations of the three dimensional model and two dimensional images from the planning phase with the patient P's airways as observed through the bronchoscope 30, and allow for the navigation phase to be undertaken with precise knowledge of the location of the sensor 44, even in portions of the airway where the bronchoscope 30 cannot reach. Further details of such a registration technique and their implementation in luminal navigation can be found in U.S. Patent Application Pub. No. 2011/0085720, the entire content of which is incorporated herein by reference, although other suitable techniques are also contemplated.

Registration of the patient P's location on the transmitter mat 56 is performed by moving the LG 32 through the airways of the patient's P. More specifically, data pertaining to locations of the sensor 44, while the locatable guide 32 is moving through the airways, is recorded using the transmitter mat 56, the reference sensors 54, and the tracking module 52. A shape resulting from this location data is compared to an interior geometry of passages of the three dimensional model generated in the planning phase, and a location correlation between the shape and the three dimensional model based on the comparison is determined, e.g., utilizing the software on the computer system 125. In addition, the software may identify non-tissue space (e.g., air filled cavities) in the three dimensional model. The software aligns, or registers, an image representing a location of sensor 44 with a three dimensional model and two dimensional images generated from the three dimension model, which are based on the recorded location data and an assumption that locatable guide 32 remains located in non-tissue space in the patient's P's airways. Alternatively, a manual registration technique may be employed by navigating the bronchoscope 30 with the sensor 44 to pre-specified locations in the lungs of the patient P, and manually correlating the images from the bronchoscope to the model data of the three dimensional model.

Following registration of the patient P to the image data and pathway plan, a user interface is displayed in the navigation software which shows the pathway that the clinician is to follow to reach the vicinity of the target with the tip of the EWC 12. One such navigation software is the superDimension™ navigation system currently sold by Medtronic PLC.

Once the EWC 12 has been successfully navigated proximate the target as depicted on the user interface, the locatable guide 32 may be unlocked from the EWC 12 and removed, leaving the EWC 12 in place as a guide channel for guiding medical devices including without limitation, optical systems, ultrasound probes, marker placement tools, biopsy tools, ablation tools (i.e., microwave ablation devices), laser probes, cryogenic probes, sensor probes, and aspirating needles to the target.

Thus, navigating a medical device to a target may be broken down into two phases. In a first phase, the EWC 12 is navigated proximate the target. In a second phase, a medical device is fed through the EWC 12 until the distal end portion of the medical device extends out of the EWC 12. The medical device is then aligned with and directed into the target using a fluoroscopic 3D reconstruction generated from a sequence of fluoroscopic images acquired as the medical device is aligned with and directed into the target.

Specifically, in the first phase, a tool, e.g., the LG 32 inside the EWC 12, is navigated to a desired location (e.g., near a target) in the patient P and re-navigating the tool using local registration correction. This may include following a pathway plan and using the EMN system described above, bronchoscopic imaging, and/or fluoroscopic imaging using the fluoroscopic imaging device 110. The fluoroscopic imaging may include performing a first fluoroscopic sweep of the EWC 12. The first fluoroscopic sweep acquires a sequence of first two dimensional (2D) fluoroscopic images at different angles as the fluoroscopic imaging device 110 rotates about the patient P. In other aspects, the intraoperative fluoroscopic imaging may be replaced with another suitable modality of intraoperative X-ray imaging, such as intraoperative cone beam computed tomography (CBCT), which may also be referred to as C-arm CT, cone beam volume CT, flat panel CT, or Digital Volume Tomography (DVT). CBCT imaging involves X-ray computed tomography where the X-rays are divergent, forming a cone. In the case of CBCT, first 2D CBCT images are acquired as a CBCT imaging device (not shown) is rotated about the patient P. Each first fluoroscopic image acquired by the fluoroscopic imaging device 110 may show radiopaque markers from a pattern of radiopaque markers disposed, for example, on the transmitter mat 56 under the patient P.

After receiving the first fluoroscopic images, a pose of the fluoroscopic imaging device 110 (e.g., a C-arm fluoroscope) is estimated for each of the first fluoroscopic images. The pose estimation may include generating a probability map. The probability map indicates the probability that each pixel of each of the first fluoroscopic images belongs to the projection of a radiopaque marker of the transmitter mat 56, which may include multiple radiopaque markers. The radiopaque markers may be in the form of a two-dimensional (2D) structure of markers. The 2D structure of markers may include multiple sphere-shaped markers arranged in a two-dimensional grid pattern.

The probability map may be generated, for example, by feeding an image into a simple marker detector, such as a Harris corner detector, which outputs a new image of smooth densities, corresponding to the probability of each pixel belonging to a marker. The probability map includes pixels or densities, which correspond to markers. In some aspects, the probability map may be downscaled (i.e., reduced in size) in order to simplify the computations.

Different candidates may be generated for the projection of the structure of markers on the image. The different candidates may be generated by virtually positioning the imaging device in a range of different possible poses. Possible poses of the fluoroscopic imaging device include 3D positions and orientations of the fluoroscopic imaging device. In some aspects, such a range may be limited according to the geometrical structure and/or degrees of freedom of the imaging device. For each possible pose, a virtual projection of at least a portion of the markers is generated, as if the fluoroscopic imaging device actually captured an image of the structure of markers while positioned at that pose.

The candidate having the highest probability of being the projection of the structure of markers on the image is identified based on the image probability map. Each candidate, i.e., a virtual projection of the structure of markers, may be overlaid or associated to the probability map. A probability score may be then determined or associated with each marker projection of the candidate. In some aspects, the probability score may be positive or negative, i.e., there may be a cost in case virtual markers projections falls within pixels of low probability. The probability scores of all of the markers' projections of a candidate may be then summed and a total probability score may be determined for each candidate. For example, if the structure of markers were a two-dimensional grid, then the projection would have a grid form. Each point of the projection grid would lie on at least one pixel of the probability map. A 2D grid candidate receives the highest probability score if its points lie on the highest density pixels, that is, if its points lie on projections of the centres of the markers on the image. The candidate having the highest probability score may be determined as the candidate which has the highest probability of being the projection of the structure of markers on the image. The pose of the imaging device while capturing the image may be then estimated based on the virtual pose of the imaging device used to generate the identified candidate.

The above-described pose estimation process is one possible pose estimation process; however, those of skill in the art will recognize that other methods and processes of pose estimation may be undertaken without departing from the scope of the disclosure. As noted above, the pose estimation process is undertaken for every image in the first fluoroscopic sweep. The result of the processing is a determination of the pose of the fluoroscopic imaging device for each image acquired.

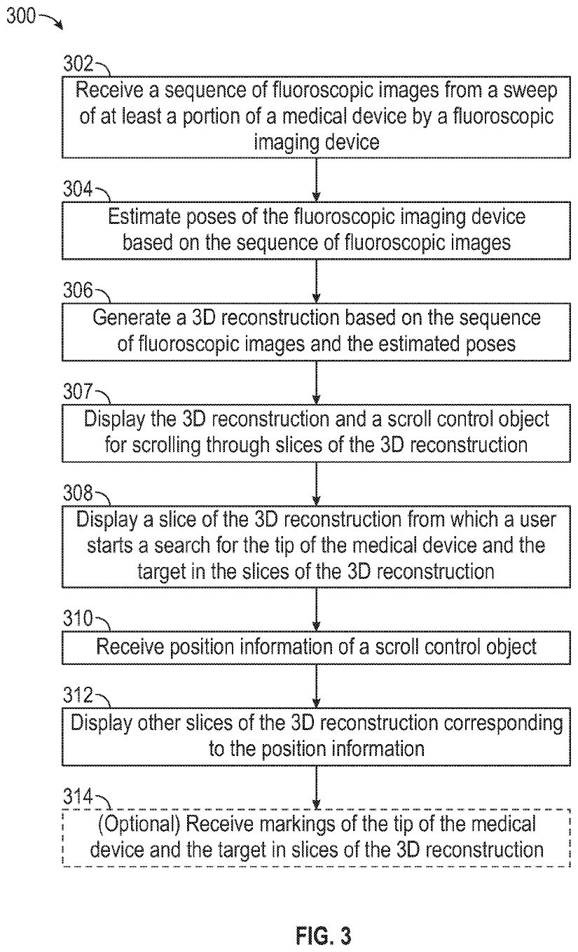

The following description of FIGS. 3-10 show examples of aspects of a workflow in the second phase using the components of system 100, including the fluoroscopic imaging device 110 and the computer system 125, to generate and display a fluoroscopic three dimensional reconstruction showing the medical device and the target, and to provide feedback to the clinician regarding the position and orientation of the tip of the medical device relative to the target. The feedback may include a textual or visual indicator of whether the tip of the medical device is aligned with the target and/or is inside the target. In aspects, the position and orientation of the tip of the medical device relative to the target is determined without the use of an EM sensor disposed on or in the medical device.

In the second phase of navigation, the medical device is passed through the EWC 12 until the medical device extends out of the EWC 12. The medical device may be a biopsy tool or a therapeutic tool (e.g., a microwave ablation catheter). The second phase of navigation also includes confirming that the medical device is aligned with and/or within the target without updating the registration or performing electromagnetic (EM) correction. The second phase of navigation also may not include adjusting or otherwise configuring the settings of the fluoroscope such as centering the fluoroscope on the catheter or setting the fluoroscopic image orientation because these functions are performed during the first phase of fluoroscopic navigation.

FIG. 3 is a flow chart of a method for verifying or confirming a position of a medical device (e.g., a biopsy tool or therapeutic tool) relative to a target (e.g., a lesion) in the second phase of fluoroscopic navigation or other suitable intraoperative X-ray navigation, e.g., CBCT navigation, by providing visual feedback to a user via a display using fluoroscopic images or other suitable intraoperative X-ray images (e.g., CBCT images) from a second sweep. At block 302, a sequence of second fluoroscopic images or other suitable second intraoperative X-ray images (e.g., second CBCT images) is obtained from a second sweep of at least a portion of a medical device by a fluoroscopic imaging device or other suitable intraoperative X-ray imaging device (e.g., a CBCT imaging device). At block 304, poses of the fluoroscopic imaging device or other suitable intraoperative imaging device (e.g., a CBCT imaging device) are estimated based on the sequence of second fluoroscopic images or other suitable second intraoperative X-ray images. At block 306, a 3D reconstruction is generated based on the sequence of second fluoroscopic images or other suitable second intraoperative X-ray images and the estimated poses.

At block 307, the 3D reconstruction and a scroll control object for scrolling through slices of the 3D reconstruction is displayed, for example, via a user interface. At block 308, a slice of the 3D reconstruction, from which a user starts a search for the tip of the medical device and the target in the slices of the 3D reconstruction, is displayed. At block 310, position information of a scroll control object, which is moved by a user during the search, is received, and, at block 312, other slices of the 3D reconstruction corresponding to different positions of the scroll control object (e.g., the clinician uses a mouse or other input device to move the scroll control object to different positions) are displayed. Then, optionally at block 314, markings of the tip of the medical device and the target in slices of the 3D reconstruction are received. Alternatively, the user can scroll through the 3D reconstruction to understand and/or analyze the relationship between the medical device and the target. If needed, the method 300 may include displaying a button or similar control object, which when selected by the user, may enable the user to mark the tip of the medical device and the target to help the user in the analysis of the relationship between the medical device and the target.

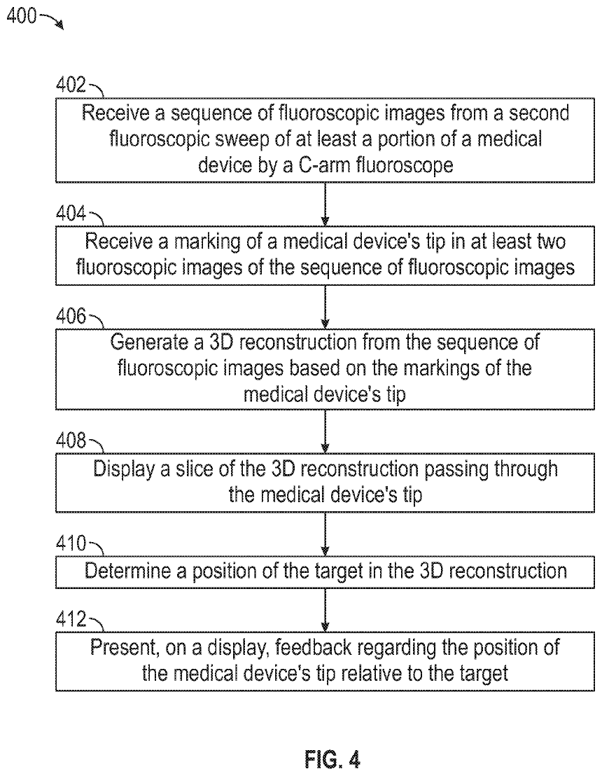

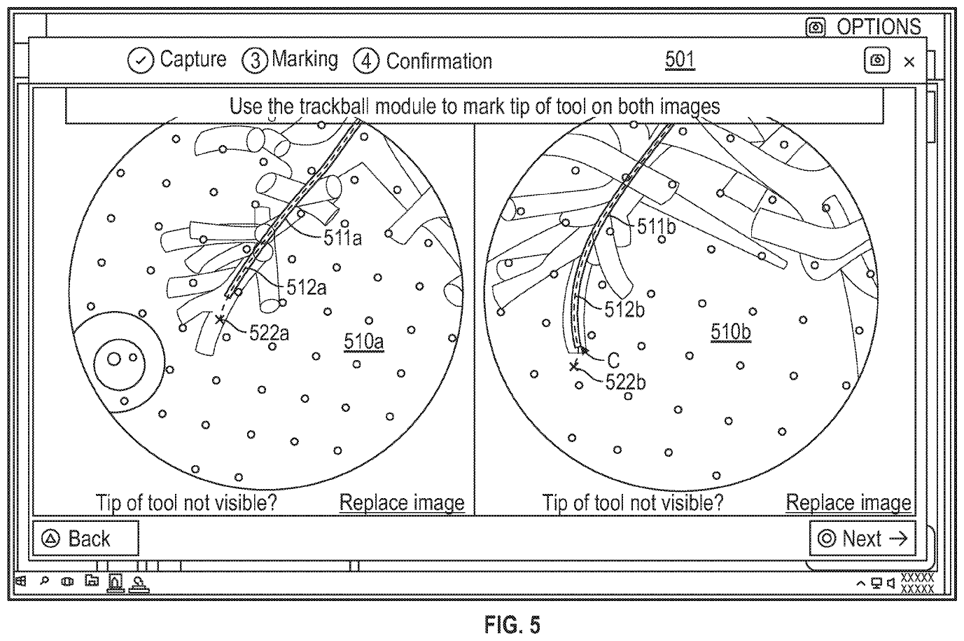

Alternatively, feedback may be provided to the user. FIG. 4 is a flowchart that illustrates a method for providing feedback to the user regarding the position of the medical device's tip relative to the target. At block 402, a sequence of second fluoroscopic images or other suitable second intraoperative X-ray images (e.g., CBCT images) is received from a second sweep of at least a portion of the medical device by a C-arm fluoroscope or other suitable intraoperative imaging device (e.g., a CBCT imaging device). At block 404, markings of a tip of the medical device, e.g., a biopsy tool, in at least two second fluoroscopic images of the sequence of second fluoroscopic images are received. In the case where CBCT imaging is employed, the marking of the medical device's tip may instead be performed in the reconstructed volume. The markings may be received from a user interface prompting the user to place marks on two second fluoroscopic images displayed in a user interface. For example, as illustrated in FIG. 5, the user interface 501 is displayed on the display of the computer system 125, in which two second fluoroscopic images 510a, 510b acquired during the second fluoroscopic sweep are presented and a clinician is prompted to mark the location of the tip of the tool 512a, 512b, which has exited the EWC 511a, 511b in each of the fluoroscopic images 510a, 510b.

The user interface 501 includes a message prompting the user to use a trackball module to mark the tip of the tool 512a, 512b in each of the second fluoroscopic images 510a, 510b. In the example of the user interface of FIG. 5, the marks 522a, 522b are placed on the tip of the tool 512a, 512b. The user interface 501 also includes messages asking whether the tip of the tool is visible in the second fluoroscopic images 510a, 510b. The user interface 501 also includes the hyperlinked text “Replace image”, which the clinician can select to replace one or both of the second fluoroscopic images 510a, 510b, if the tip of the tool 512a, 512b is not visible or is difficult for the clinician to see. Alternatively, the method 400 may include segmenting the tip of the medical device in two second fluoroscopic images and determining the positions of the segmented tip of the medical device in the two second fluoroscopic images based on the segmenting of the tip of the medical device. Segmenting the tip of the medical device may include segmenting the tip of the medical device using a neural network, e.g., a convolutional neural network.

While marking of the medical device's tip may be performed either in the 3D reconstruction according to the method 300 of FIG. 3 or in the 2D images according to the method of FIG. 4, the marking of the medical device's tip in the methods 300, 400 of FIGS. 3 and 4 may be performed in the 3D reconstruction and/or in one or more of the 2D images. For example, the method of FIG. 3 may additionally or alternatively include marking or receiving a marking in one or more of the 2D images. Likewise, in another example, the method of FIG. 4 may additionally or alternatively include marking or receiving a marking of the medical device's tip in the 3D reconstruction. Additionally, or alternatively, the coordinates of the medical device's tip coordinates may be automatically estimated, using, for example, a suitable image recognition process, such as a segmenting algorithm.

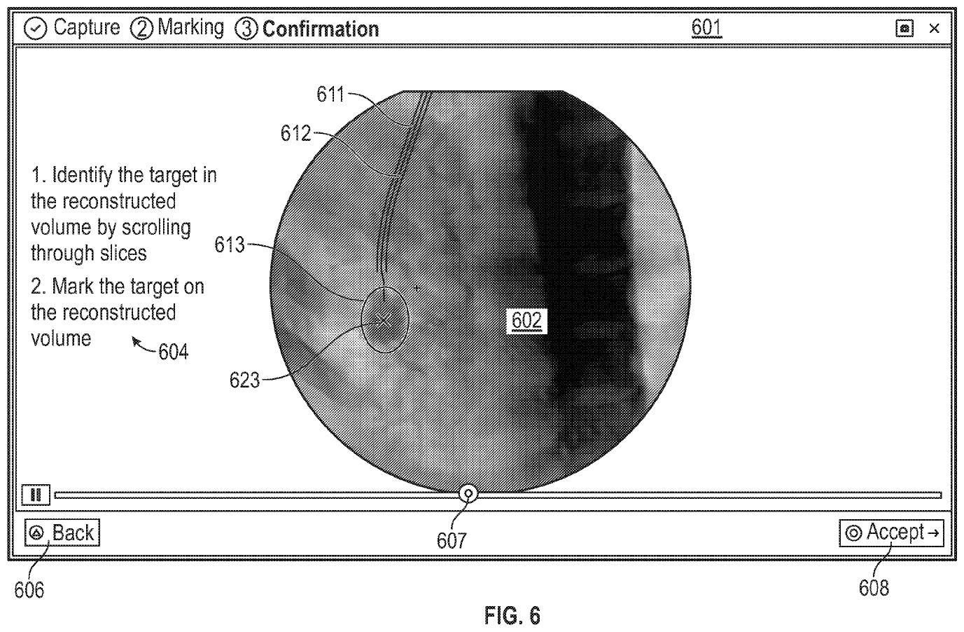

At block 406, a second 3D reconstruction of the second fluoroscopic images is generated based on the markings of the medical device's tip. At block 408, a slice of the second 3D reconstruction passing through the medical device's tip is displayed. At block 410, a position of the target in the second 3D reconstruction is determined. The position of the target in the second 3D reconstruction may be determined by using the user interface 601 illustrated in FIG. 6.

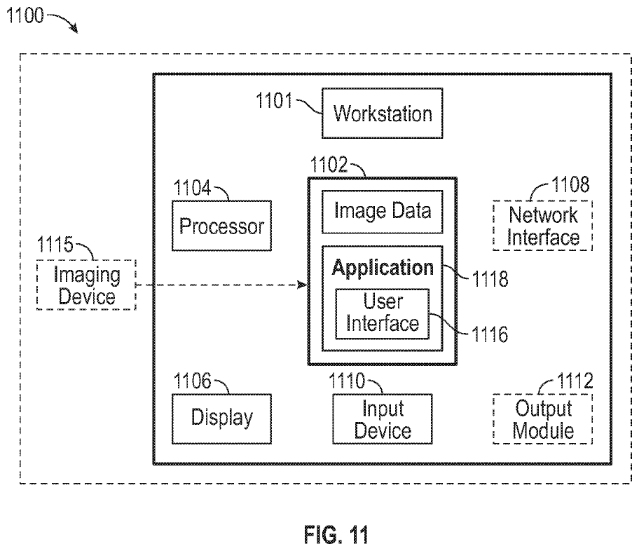

A user interface may be displayed on the display of the computer system 125, in which a second 3D reconstruction is presented to a clinician and the clinician is asked to identify a position of a target in the second fluoroscopic 3D reconstruction. For example, as illustrated in FIG. 6, user interface 601 is displayed on the display of the computer system 125, in which the second fluoroscopic 3D reconstruction 602 is presented and a clinician is asked by the instructions 604 to identify the target by scrolling through slices of the second 3D reconstruction and mark a position of the target in the second fluoroscopic 3D reconstruction 602. The clinician may then use an input device 1110 (FIG. 11), e.g., a trackball or trackpad, to place a marking 623 at one position on the target. Alternatively, the clinician may use the input device 1110 to place an ellipse 613 on the target to indicate the approximate edges of the target. The input device 1110 may be used by the clinician to change the size and/or shape of the ellipse 613 so that the clinician can ensure that the ellipse 613 closely approximate the edges of the target.

Additionally, or alternatively, the position of the target may be obtained by registering the first 3D reconstruction with the second 3D reconstruction using a suitable method, such as a mutual information method. Since the user marked the target in the first reconstruction, the system knows the location of the target in the second reconstruction.

The user interface 601 also includes a scroll button 607, which the clinician may select and move left or right to change to a different slice of the second 3D reconstruction. The clinician may move the scroll button 607 to search for a slice of the second 3D reconstruction that gives the clinician the best view of the catheter 611, the tool 612, and the target, such that the clinician can accurately place the marking 623.

Additionally, or alternatively, the computer system 125 may execute an application that automatically segments the target to determine the position of the target. The segmentation may be performed using a suitable method such as a convolutional neural network (CNN).

The user interface 601 also includes a back button 606, which the clinician can select to return to previous screens of the user interface 601. For example, the clinician can select the “Back” button 606, which returns the user interface 601 to a “capture” screen, which may be used by the clinician to recapture second fluoroscopic images if the clinician finds that the existing second fluoroscopic images have poor quality. The user interface 601 also includes an “Accept” button 608, which the clinician can select to confirm placement of the markings 621-623 on the second fluoroscopic 3D reconstruction 602.

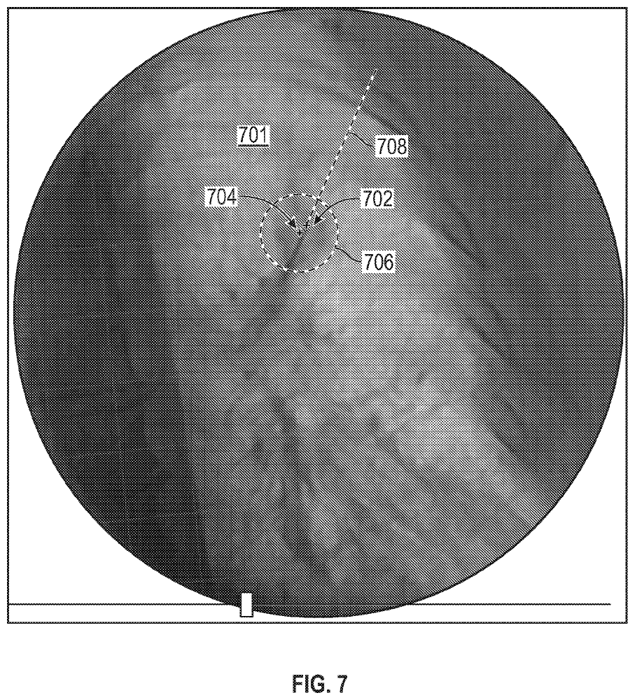

After the clinician selects the accept button 608, feedback regarding the position of the medical device's tip relative to the target is presented on the display at block 412. The feedback may include augmenting the marking of the target or the marking of the medical device to show that the medical device is inside or outside the target. For example, as illustrated in FIG. 7, a crosshairs symbol 704 indicating the center of the target and an ellipse, e.g., a circle 706, indicating the largest size of the target may be overlaid on the 3D reconstruction 701. Additionally, or alternatively, a trajectory 708 of the tip 702 of medical device may be overlaid on the 3D reconstruction 701. The trajectory 708 may be determined based on the orientation of the medical device.

The orientation of the medical device may be determined according to a suitable method known to a person skilled in the art. For example, the user may draw a line starting from the tip 702 of the medical device and going backward along the medical device. Then, the user's line is used to compute the 3D orientation of the medical device. Alternatively, after determining the position of the tip 702 of the medical device, the orientation of the medical device may be estimated in multiple fluoroscopic 2D images. For example, the orientation of the medical device may be estimated in multiple fluoroscopic 2D images based on gradients analysis in the vicinity of the tip 702 of the medical device. Then, the 2D orientations of the medical device can be combined into a 3D orientation of the tip 702 of the medical device. In one aspect, determining the position of the tip 702 of the medical device may include estimating the position of the tip 702. The feedback may include presenting a message indicating that the medical device is inside or outside the target.

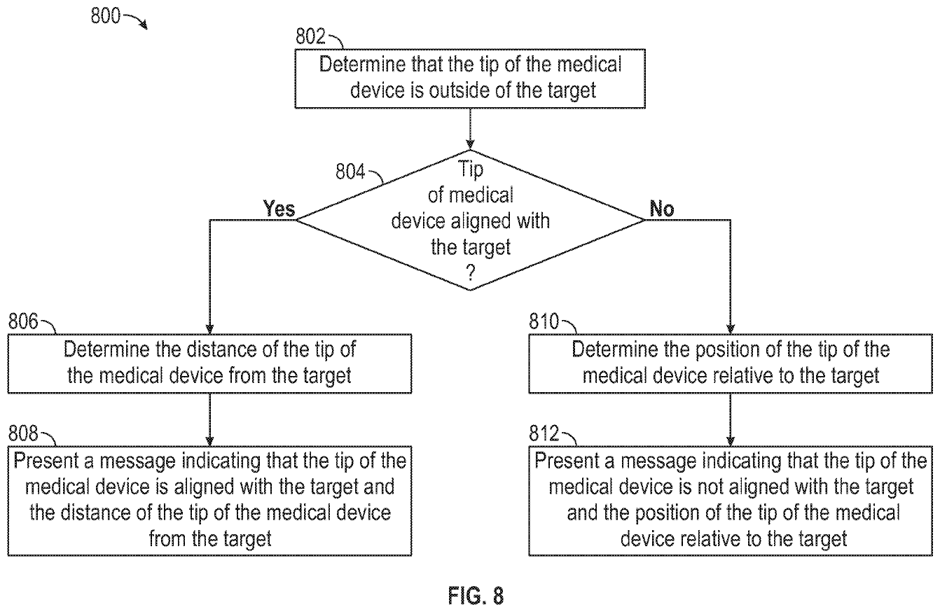

FIG. 8 is a flowchart that illustrates an example of a method 800 of providing visual feedback to a user via a display. At block 802, the method 800 determines that the tip of the medical device is outside of the target. The method 800 may determine that the tip of the medical device is outside of the target based on preoperative target information, which is registered to the fluoroscopic 3D reconstruction including the tip of the medical device. The preoperative target information may be obtained from the planning stage during which the center coordinates of the target are manually or automatically collected. The target information from the planning stage may be a simple ellipsoid that encapsulates the target in a preoperative CT image and that is marked by the user. Alternatively, the method 800 may include segmenting the target in the preoperative CT image to obtain a more accurate 3D representation of the target.

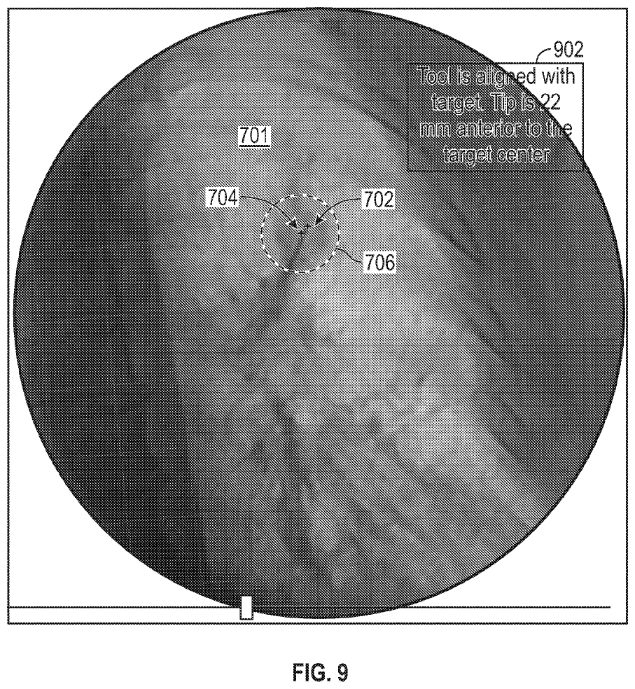

At block 804, the method 800 determines whether the tip of the medical device is aligned with the target. If the tip of the medical device is aligned with the target, the distance of the tip of the medical device from the target is determined at block 806. Then, a message indicating that the tip of the medical device is aligned with the target and the distance of the tip of the medical device from the target is presented on a display at block 808. For example, as illustrated in FIG. 9, a message box 902 including the text “Tool is aligned with target. Tip is 22 mm anterior to the target center,” may be displayed overlaid on the displayed 3D reconstruction 701. If the tip of the medical device is not aligned with the target, the position of the medical device's tip relative to the target is determined at block 810. Then, a message indicating that the medical device's tip is not aligned with the target and the position of the medical device's tip relative to the target is presented on a display at block 812.

In aspects, the method 800 may include receiving preoperative computed tomography (CT) images of the target, constructing a 3D model of the target based on the preoperative CT images, and overlaying the 3D model of the target on the 3D reconstruction. The method may include registering the preoperative CT images with the 3D reconstruction, determining a position of the target in the 3D reconstruction based on the registering, yielding a determined position of the target, and overlaying the 3D model of the target on the 3D reconstruction based on the determined position of the target.

The method 800 may include constructing a 3D model of the medical device based on the sequence of fluoroscopic images, and overlaying the 3D model of the medical device on the 3D reconstruction. The method 800 may include receiving preoperative computed tomography (CT) images of the target, segmenting the target from the preoperative CT images, yielding a segmented target, and overlaying the segmented target on the 3D reconstruction. The method 800 may include receiving preoperative computed tomography (CT) images of a lung, receiving at least one marking of the target on the preoperative CT images, and overlaying the at least one marking of the target on the 3D reconstruction. The at least one marking of the target may include a shape of the target or may represent a size of the target. In some aspects, the medical device does not include a position sensor.

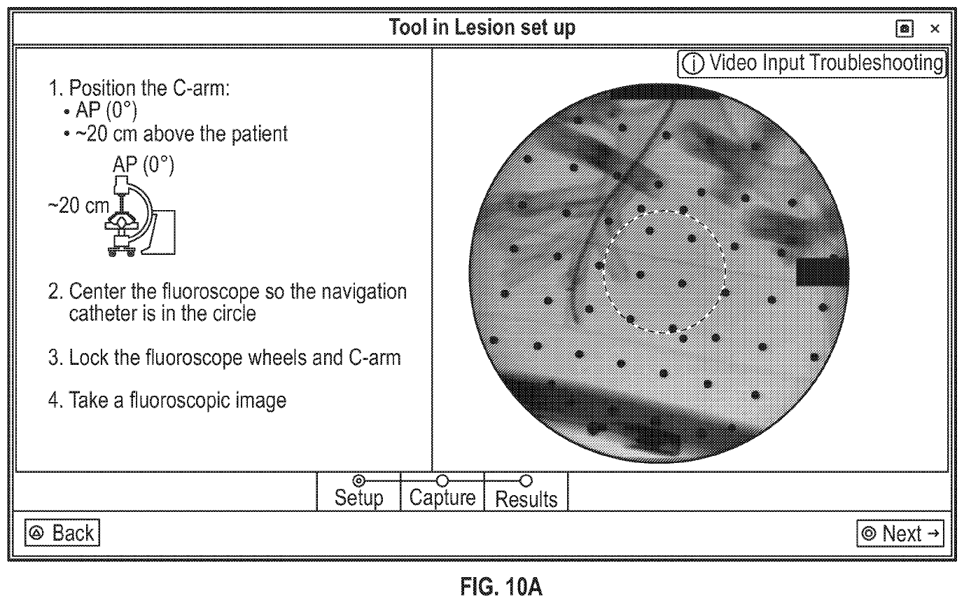

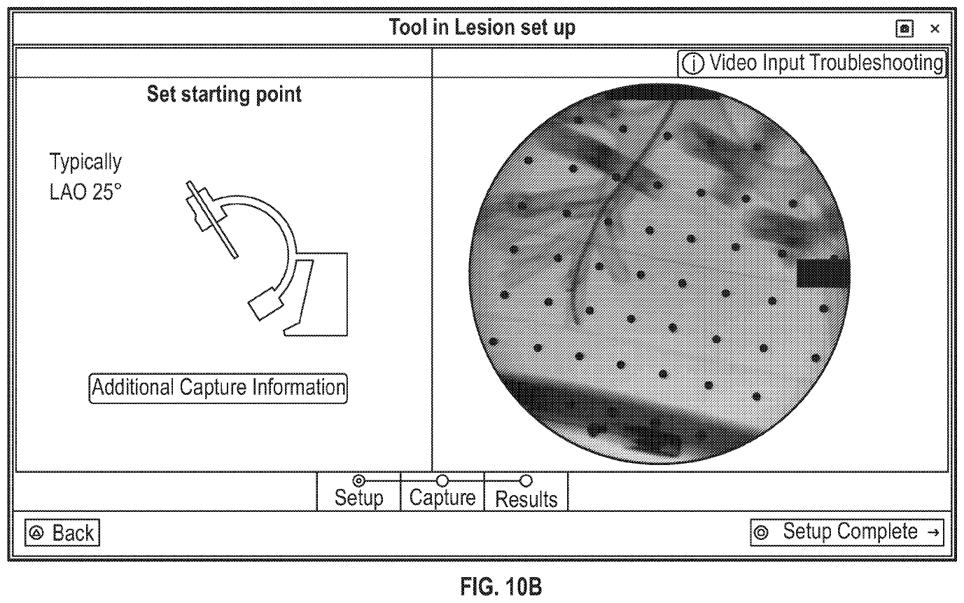

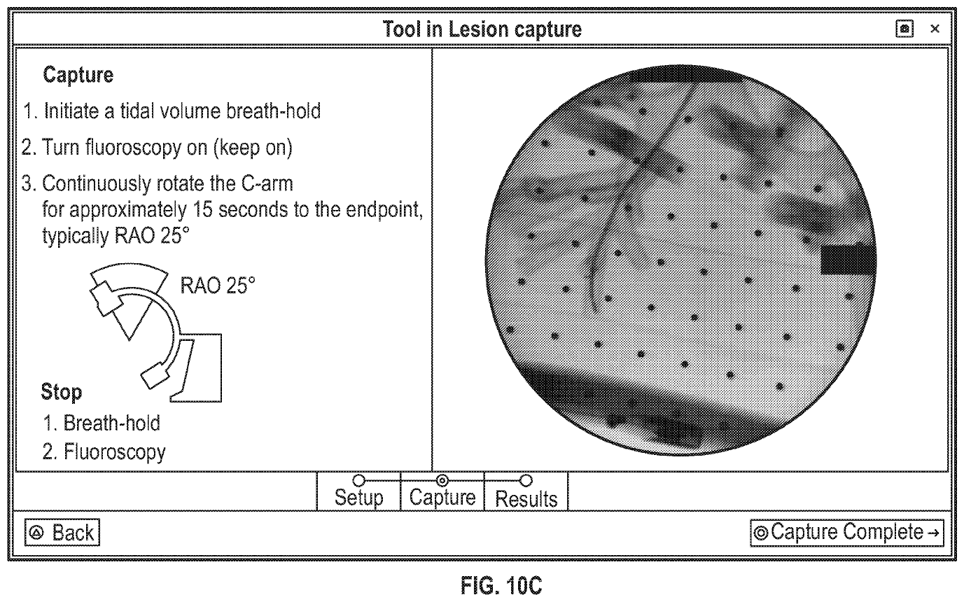

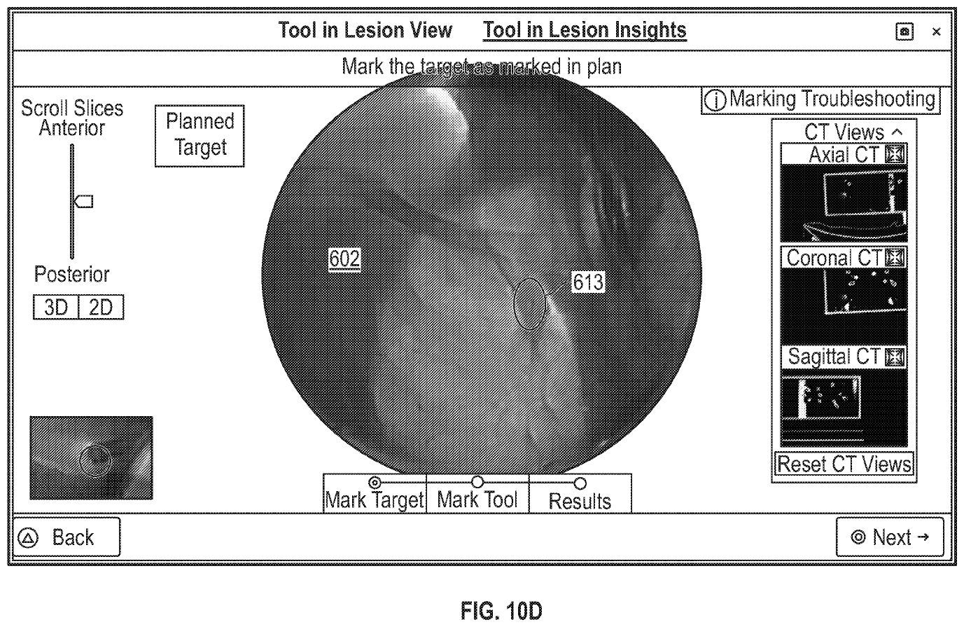

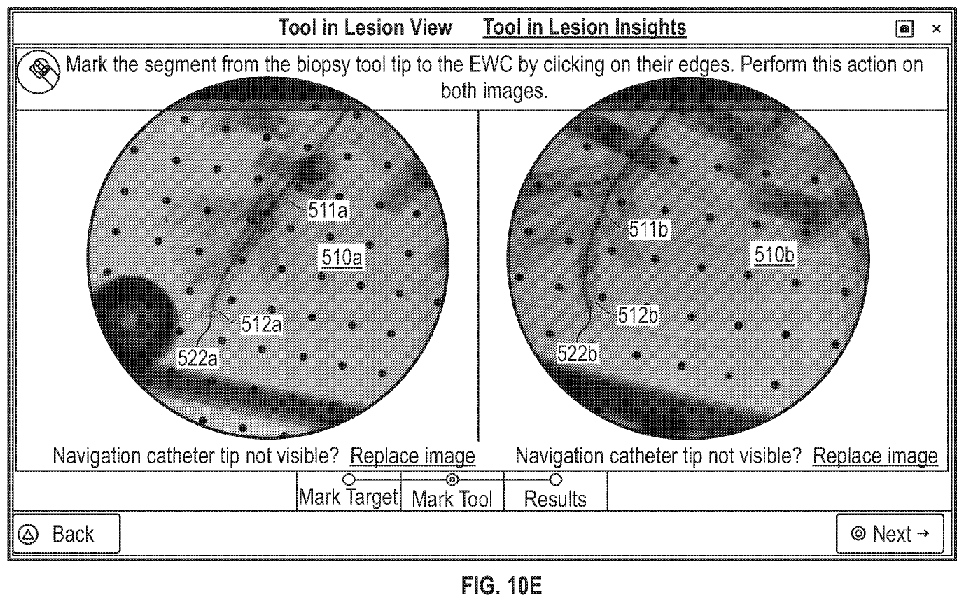

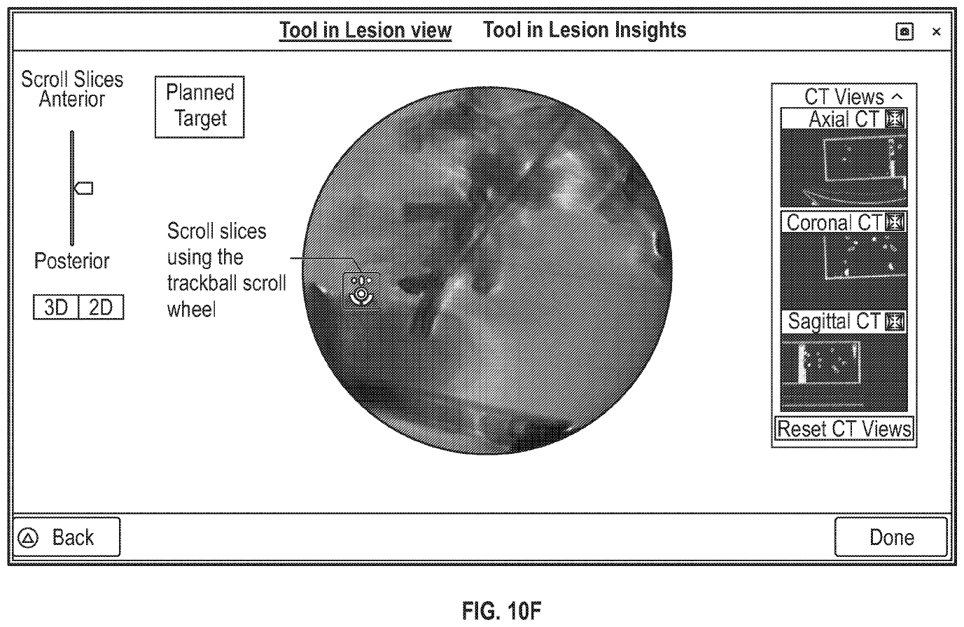

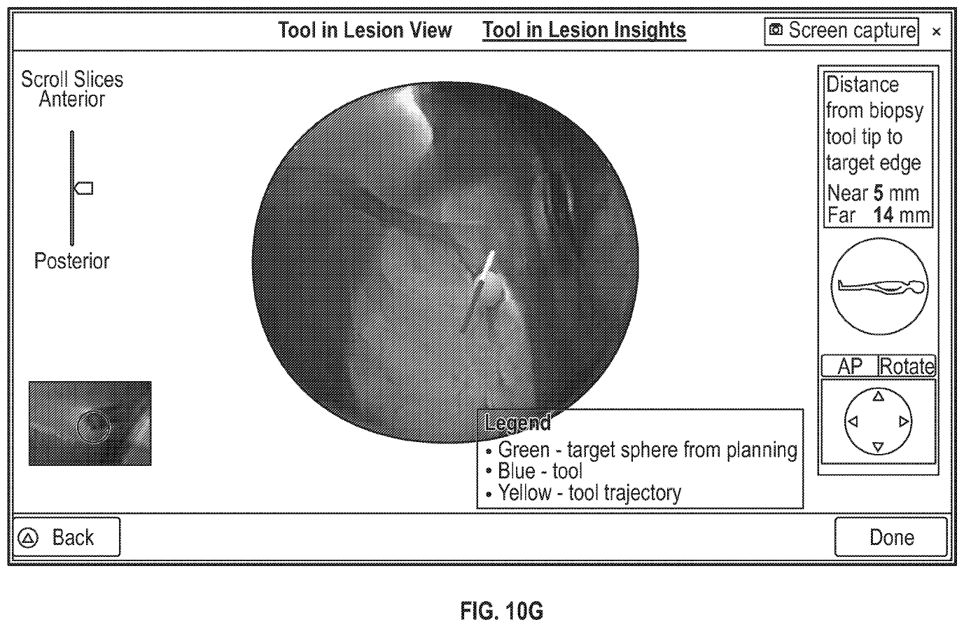

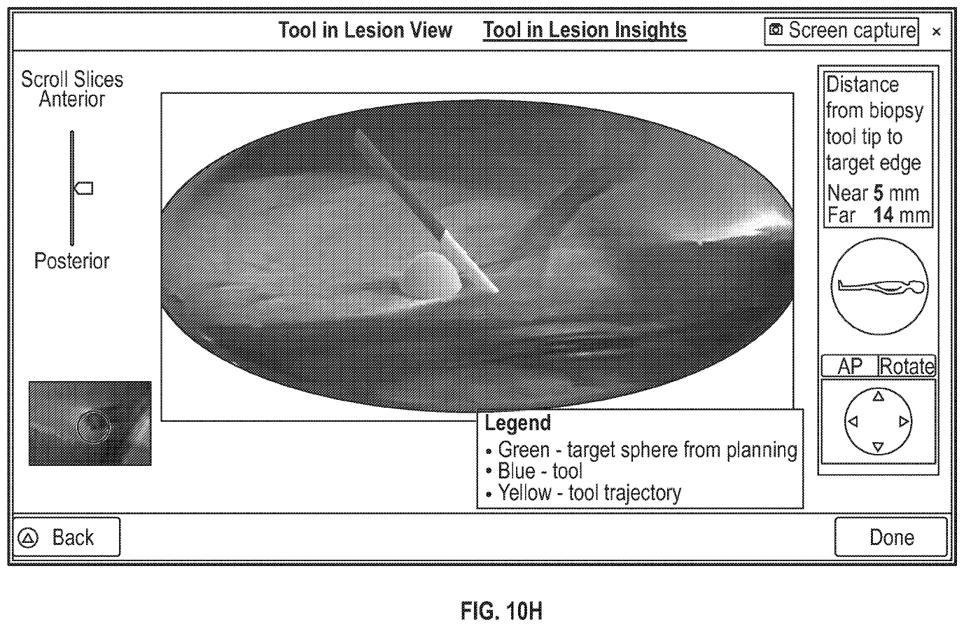

FIGS. 10A-10H are diagrams that illustrate other examples of user interfaces that implement aspects of the methods described herein. FIGS. 10A-10C show user interfaces for setting up a fluoroscope and capturing fluoroscopic images. FIGS. 10D and 10E show other examples of user interfaces similar to the user interfaces of FIGS. 5 and 6 for applying an ellipse 613 or other suitable marking on the target in a slice of a second fluoroscopic 3D reconstruction 602 and for applying marks 522a, 522b to a tip of a tool 512a, 512b protruding from an EWC 511a, 511b in two fluoroscopic images 510a, 510b. FIG. 10F shows an example of a user interface that allows a clinician to scroll through slices of the 3D reconstruction to visually identify a tool's tip and a target. FIGS. 10G and 10H show another example of a user interface that augments the 3D reconstruction with representations of a target (e.g., a target sphere from planning), a tool, and a tool trajectory. As shown in FIGS. 10G and 10H, the user interface may include user controls for rotating the augmented 3D reconstruction to visualize the tool and the tool trajectory relative to the target.

Reference is now made to FIG. 11, which is a diagram of a system 1100 configured for use with the methods of the disclosure. The system 1100 may include a workstation 1101, which may be optionally connected to a fluoroscopic imaging device 110 (FIG. 1). In some aspects, the workstation 1101 may be coupled with the fluoroscope 1115, directly or indirectly, e.g., by wireless communication. The workstation 1101 may include a memory 1102, a processor 1104, a display 1106 and an input device 1110. Processor 1104 may include one or more hardware processors. The workstation 1101 may optionally include an output module 1112 and a network interface 1108. The memory 1102 may store an application 1118 and image data 1114. The application 1118 may include instructions executable by the processor 1104 for executing the methods of the disclosure including the methods of FIGS. 3, 4, and 8.

The application 1118 may further include a user interface 1116. Image data 1114 may include the CT scans, the sequence of fluoroscopic images, the fluoroscopic 3D reconstructions and/or any other imaging information. The processor 1104 may be coupled with the memory 1102, display 1106, the input device 1110, the output module 1112, the network interface 1108 and the fluoroscope 1115. The workstation 1101 may be a stationary computer system, such as a personal computer, or a portable computer system such as a tablet computer. The workstation 1101 may embed multiple computer devices.

The memory 1102 may include any non-transitory computer-readable storage media for storing data and/or software including instructions that are executable by the processor 1104 and which control the operation of the workstation 1101 and, in some aspects, may also control the operation of the fluoroscope 1115. The fluoroscopic imaging device 110 may be used to capture a sequence of fluoroscopic images based on which the fluoroscopic 3D reconstruction is generated and to capture a live 2D fluoroscopic view according to this disclosure. In an aspect, the memory 1102 may include one or more storage devices such as solid-state storage devices, e.g., flash memory chips. Alternatively, or in addition to the one or more solid-state storage devices, the memory 1102 may include one or more mass storage devices connected to the processor 1104 through a mass storage controller (not shown) and a communications bus (not shown).

Although the description of computer-readable media contained herein refers to solid-state storage, it should be appreciated by those skilled in the art that computer-readable storage media can be any available media that can be accessed by the processor 1104. That is, computer readable storage media may include non-transitory, volatile and non-volatile, removable and non-removable media implemented in any method or technology for storage of information such as computer-readable instructions, data structures, program modules or other data. For example, computer-readable storage media may include RAM, ROM, EPROM, EEPROM, flash memory or other solid-state memory technology, CD-ROM, DVD, Blu-Ray or other optical storage, magnetic cassettes, magnetic tape, magnetic disk storage or other magnetic storage devices, or any other medium which may be used to store the desired information, and which may be accessed by the workstation 1101.

The application 1118 may, when executed by the processor 1104, cause the display 1106 to present the user interface 1116. The user interface 1116 may be configured to present to the user a single screen including a three-dimensional (3D) rendering of the tool, the lesion, and/or the catheter of this disclosure. The user interface 1116 may be further configured to display the lesion in different colors depending on whether the tool tip is aligned with the lesion in three dimensions.

The network interface 1108 may be configured to connect to a network such as a local area network (LAN) consisting of a wired network and/or a wireless network, a wide area network (WAN), a wireless mobile network, a Bluetooth network, and/or the Internet. Network interface 1108 may be used to connect between the workstation 1101 and the fluoroscope 1115. The network interface 1108 may be also used to receive the image data 1114. The input device 1110 may be any device by which a user may interact with the workstation 1101, such as, for example, a mouse, keyboard, foot pedal, touch screen, and/or voice interface. The output module 1112 may include any connectivity port or bus, such as, for example, parallel ports, serial ports, universal serial busses (USB), or any other similar connectivity port known to those skilled in the art.

From the foregoing and with reference to the various figures in the drawings, those skilled in the art will appreciate that certain modifications can also be made to the disclosure without departing from the scope of the disclosure. For example, although the systems and methods are described as usable with an EMN system for navigation through a luminal network such as the lungs, the systems and methods described herein may be utilized with systems that utilize other navigation and treatment devices such as percutaneous devices. Additionally, although the above-described systems and methods are described as used within a patient's luminal network, it is appreciated that the above-described systems and methods may be utilized in other target regions such as the liver. Further, the above-described systems and methods are also usable for transthoracic needle aspiration procedures.

Detailed aspects of the disclosure are disclosed herein. However, the disclosed aspects are merely examples of the disclosure, which may be embodied in various forms and aspects. Therefore, specific structural and functional details disclosed herein are not to be interpreted as limiting, but merely as a basis for the claims and as a representative basis for teaching one skilled in the art to variously employ the disclosure in virtually any appropriately detailed structure.

As can be appreciated a medical instrument such as a biopsy tool or an energy device, such as a microwave ablation catheter, that is positionable through one or more branched luminal networks of a patient to treat tissue may prove useful in the surgical arena and the disclosure is directed to systems and methods that are usable with such instruments, tools, and devices. Access to luminal networks may be percutaneous or through natural orifice using navigation techniques. Additionally, navigation through a luminal network may be accomplished using image-guidance. These image-guidance systems may be separate or integrated with the biopsy tool or energy device or a separate access tool and may include MRI, CT, fluoroscopy, ultrasound, electrical impedance tomography, optical, and/or device tracking systems. Methodologies for locating the access tool include EM, IR, echolocation, optical, and others. Tracking systems may be integrated to an imaging device, where tracking is done in virtual space or fused with preoperative or live images.

In some cases, the treatment target may be directly accessed from within the lumen, such as for the treatment of the endobronchial wall for COPD, Asthma, lung cancer, etc. In other cases, the biopsy tool, the energy device and/or the additional access tool may be required to pierce the lumen and extend into other tissues to reach the target, such as for the treatment of disease within the parenchyma. Final localization and confirmation of energy device or tool placement may be performed with imaging and/or navigational guidance using a standard fluoroscopic imaging device incorporated with methods and systems described above.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A system comprising:
a computer system including a processor and a display configured to display a graphical user interface, and a computer readable storage medium storing thereon instructions that when executed by the processor cause the processor to:
receive a sequence of intraoperative X-ray images, each intraoperative X-ray image including at least a portion of a medical device and a target;
receive a marking of a tip of the medical device in at least two of the sequence of intraoperative X-ray images;
construct a three-dimensional (3D) reconstruction based on the sequence of intraoperative X-ray images and the markings of the tip of the medical device, the 3D reconstruction including the medical device and the target;
display a slice of the 3D reconstruction passing through the tip of the medical device;
determine a position of the target in the 3D reconstruction; and
present feedback on the position of the tip of the medical device relative to the target.

2. The system of claim 1, wherein the instructions, when executed by the processor, further cause the processor to augment the marking of the target or the marking of the medical device to show that the medical device is inside or outside the target.

3. The system of claim 1, wherein the instructions, when executed by the processor, further cause the processor to present a message indicating that the medical device is inside or outside the target.

4. The system of claim 1, wherein the instructions, when executed by the processor, further cause the processor to:
determine that the tip of the medical device is outside of the target;
in response to determining that the tip of the medical device is outside of the target, determine whether the tip of the medical device is aligned with the target; and
present a message indicating whether the tip of the medical device is aligned with the target.

5. The system of claim 1, wherein the instructions, when executed by the processor, further cause the processor to:
determine that the tip of the medical device is outside of the target;
in response to determining that the tip of the medical device is outside of the target, determine a distance of the tip of the medical device from the target or the position of the tip of the medical device relative to the target; and
present a message indicating the distance of the tip of the medical device from the target or the position of the tip of the medical device relative to the target.

6. The system of claim 1, wherein the instructions, when executed by the processor, further cause the processor to:
receive preoperative computed tomography (CT) images of the target;
construct a 3D model of the target based on the preoperative CT images; and
overlay the 3D model of the target on the 3D reconstruction.

7. The system of claim 6, wherein the instructions, when executed by the processor, further cause the processor to:
register the preoperative CT images with the 3D reconstruction;
determine a position of the target in the 3D reconstruction based on the registering, yielding a determined position of the target; and
overlay the 3D model of the target on the 3D reconstruction based on the determined position of the target.

8. The system of claim 1, wherein the instructions, when executed by the processor, further cause the processor to:
construct a 3D model of the medical device based on the sequence of intraoperative X-ray images; and
overlay the 3D model of the medical device on the 3D reconstruction.

9. The system of claim 1, wherein the instructions, when executed by the processor, further cause the processor to:
receive preoperative computed tomography (CT) images of the target;
segment the target from the preoperative CT images, yielding a segmented target; and
overlay the segmented target on the 3D reconstruction.

10. The system of claim 1, wherein the instructions, when executed by the processor, further cause the processor to:
receive preoperative computed tomography (CT) images of a lung;
receive at least one marking of the target on the preoperative CT images; and
overlay the at least one marking of the target on the 3D reconstruction.

11. The system of claim 10, wherein the at least one marking of the target represents a size or a shape of the target.

12. The system of claim 1, wherein the sequence of intraoperative X-ray images are fluoroscopic images or cone beam CT images.

13. The system of claim 1, wherein the medical device does not include a position sensor.

14. The system of claim 1, wherein the medical device is a biopsy tool.

15. A method comprising:
receiving a sequence of intraoperative X-ray images;
receiving a marking of a tip of a medical device in at least two of the sequence of intraoperative X-ray images;
generating a three-dimensional (3D) reconstruction based on the sequence of intraoperative X-ray images and the markings of the tip of the medical device, the 3D reconstruction including the at least a portion of the medical device and a target;
displaying a slice of the 3D reconstruction passing through the tip of the medical device;
determining a position of the target in the 3D reconstruction; and
presenting feedback on the position of the tip of the medical device relative to the target.

16. The method of claim 15, further comprising augmenting the marking of the target or the marking of the medical device to show that the medical device is inside or outside the target.

17. The method of claim 15, further comprising presenting a message indicating that the medical device is inside or outside the target.

18. The method of claim 15, further comprising:
determining that the tip of the medical device is outside of the target;
determining that the tip of the medical device is aligned with the target in response to determining that the tip of the medical device is outside of the target;
determining a distance of the tip of the medical device from the target in response to determining that the tip of the medical device is aligned with the target; and
presenting a message indicating that the tip of the medical device is aligned with the target and the distance of the tip of the medical device from the target.

19. The method of claim 15, further comprising:
determining that the tip of the medical device is outside of the target;
determining that the tip of the medical device is not aligned with the target in response to determining that the tip of the medical device is outside of the target;
determining the position of the tip of the medical device relative to the target in response to determining that the tip of the medical device is not aligned with the target; and
presenting a message indicating that the tip of the medical device is not aligned with the target and the position of the tip of the medical device relative to the target.

20. A system comprising:
a computer system including a processor and a display configured to display a graphical user interface, and a computer readable storage medium storing thereon instructions that when executed by the processor cause the processor to:
receive a sequence of intraoperative X-ray images from an X-ray imaging device, each intraoperative X-ray image including at least a portion of a medical device and a target;
estimate a pose of the X-ray imaging device based on the sequence of intraoperative X-ray images, yielding an estimated pose;
generate a three-dimensional (3D) volume based on the sequence of intraoperative X-ray images and the estimated pose, the 3D volume including the at least a portion of the medical device and the target;
display a slice of the 3D volume from which a user starts a search for a tip of the medical device and the target in the slices of the 3D volume;
receive position information of a scroll control object; and
display other slices of the 3D volume corresponding to the position information.
